(19) United States
(12) Patent Application Publication
(10) Pub. No.: US 2026/0093979 A1
(43) Pub. Date: Apr. 2, 2026

(54) METHODS FOR TRAINING FOUNDATION MODELS FOR PROCESSING OPTICAL PHYSIOLOGICAL SIGNALS

(71) Applicant: Nokia Solutions and Networks Oy, Espoo (FI)

(72) Inventors: Dimitrios SPATHIS, Cambridge (GB); Mohammad MALEKZADEH, Cambridge (GB); Arvind THANGA CHELLAPPA PILLAI, Lebanon, NH (US)

(73) Assignee: Nokia Solutions and Networks Oy, Espoo (FI)

(21) Appl. No.: 19/325,955

(22) Filed: Sep. 11, 2025

Related U.S. Application Data
(60) Provisional application No. 63/700,802, filed on Sep. 30, 2024.

(51) Int. Cl. G06N 3/08; G06N 3/045; G16H 50/30

(52) U.S. Cl. CPC G06N 3/08; G06N 3/045; G16H 50/30

(57) ABSTRACT

Provided is a method of training an artificial intelligence (AI) model including providing training data to the AI model, the AI model including a plurality of machine learning models and the training data including Photoplethysmography (PPG) data based on PPG signals obtained from a plurality of individuals, and training the AI model includes using the training data to determine one or more outputs based on the PPG data.

### CROSS-REFERENCE TO RELATED APPLICATION(S)

This application claims priority to U.S. Provisional Application No. 63/700,802, filed on Sep. 30, 2024, in the United States Patent and Trademark Office (USPTO), the disclosure of which is incorporated herein by reference in its entirety.

### TECHNICAL FIELD

Example embodiments of inventive concepts are generally related to photoplethysmography (PPG), and more particularly, to methods of using and training an artificial intelligence (AI) model to process PPG data including PPG signals.

### BACKGROUND

Photoplethysmography (PPG) is a widely used non-invasive technique for monitoring cardiovascular health and other physiological signals, with widespread use in both clinical and consumer health applications.

### SUMMARY

The scope of protection sought for various example embodiments are set out by the independent claims. Some example embodiments and/or features, if any, described in this specification that do not fall under the scope of the independent claims are to be interpreted as examples useful for understanding various example embodiments.

Despite its widespread adoption, machine learning with PPG data may be relatively difficult with regard to data annotation. Unlike image, audio, or text data, where labels can often be inferred with relative ease, annotating PPG signals typically requires domain-specific expertise and may be relatively labor-intensive. This issue may be further exacerbated in consumer health applications, where the variety of recording conditions and subject heterogeneity may make consistent labeling even more difficult. PPG signals may also be susceptible to noise and motion artifacts introduced by poor sensor placement or ambient light, which may further complicate the development of robust models. This variability in PPG data included in PPG datasets, arising from factors like skin tone and body composition, may also contribute to the difficulties in building generalizable machine learning approaches. As a result, existing PPG datasets are often small, task-specific, and limited in their generalizability.

Some example embodiments provide methods of using and training an artificial intelligence (AI) model to process PPG data including PPG signals.

In at least one example embodiment, a method of training an artificial intelligence (AI) model is provided, the method includes providing training data to the AI model, the AI model including a plurality of machine learning models and the training data including Photoplethysmography (PPG) data based on PPG signals obtained from a plurality of individuals, and training the AI model, using the training data, to determine one or more outputs based on the PPG data.

In at least one example embodiment, the training includes training the AI model, using the training data, to determine one or more loss functions, and training the AI model, using the one or more loss functions, to determine the one or more outputs.

In at least one example embodiment, the AI model is configured such that the one or more outputs include a first output, a second output, and a third output, the one or more loss functions include a first loss function, a second loss function, and a third loss function, the first loss function is based on a first true value and the first output, the second loss function is based on a second true value and the second output, and the third loss function is based on a third true value and the third output.

In at least one example embodiment, the training the AI model, using the one or more loss functions, to determine the one or more outputs includes continuously adjusting parameters of the plurality of machine learning models until the first output equals the first true value, the second output equals the second true value, and the third output equals the third true value.

In at least one example embodiment, the first output is based on data representing an inflection point area ratio (IPA) morphology metric of the PPG data, the second output is based on data representing a stress-induced vascular response index (sVRI) morphology metric of the PPG data, and the third output is based on data representing a signal quality index (SQI) morphology metric of the PPG data.

In at least one example embodiment, the plurality of machine learning models are configured to receive the PPG data and output the one or more outputs, the one or more outputs including a first output, a second output, and a third output.

In at least one example embodiment, the plurality of machine learning models include a first machine learning model including a first neural network configured to receive the PPG data and output the first output, a second machine learning model including a second neural network configured to receive the PPG data and output the second output, and a third machine learning model including a third neural network configured to receive the PPG data and output the third output.

In at least one example embodiment, the training the AI model, using the training data, to determine one or more outputs based on the PPG data includes receiving, by an encoder, the PPG data including the PPG signals, processing, by the encoder, the PPG data to generate and output a first embedding of PPG data, generating, by the first machine learning model, the first output based on the first embedding of PPG data, processing, by the second machine learning model, the first embedding of PPG data to generate a second embedding of PPG data, generating, by the second machine learning model, the second output based on the second embedding of PPG data, and generating, by the third machine learning model, the third output based on the first embedding of PPG data.

In at least one example embodiment, the method of training the artificial intelligence (AI) model further includes processing the PPG data to filter noise in the PPG signals before generating the first embedding of PPG data.

In at least one example embodiment, the AI model is configured to link the PPG data to one or more of health conditions based on processing the PPG data.

In at least one example embodiment, a processing device is provided, the processing device including a memory storing a program of instructions, and a processor coupled to the memory, the processor configured to execute the program of instructions to implement an artificial intelligence (AI) model trained on training data including Photoplethysmography (PPG) data based on PPG signals, the AI model including a plurality of machine learning models and trained to receive the PPG data including the PPG signals, and generate an output corresponding to the PPG data.

In at least one example embodiment, the AI model is trained to determine one or more morphology metrics of the PPG data, and generate the output based on the one or more morphology metrics of the PPG data.

In at least one example embodiment, the one or more morphology metrics of the PPG data includes an inflection point area ratio (IPA) morphology metric of the PPG data, a stress-induced vascular response index (sVRI) morphology metric of the PPG data, and a signal quality index (SQI) morphology metric of the PPG data.

In at least one example embodiment, the training the AI model includes receiving, by an encoder, the PPG data including the PPG signals, processing, by the encoder, the PPG data to generate and output a first embedding of PPG, generating, by a first machine learning model, a first output and a first loss function, the first output based on the first embedding of PPG data, generating, by a second machine learning model, a second output and a second loss function, the second output based on a second embedding of PPG data, the second embedding of PPG data generated by the second machine learning model based on the first embedding of PPG data, and generating, by a third machine learning model, a third output and a third loss function, the third output based on the first embedding of PPG data, wherein the first loss function is based on a first true value and the first output, the second loss function is based on a second true value and the second output, and the third loss function is based on a third true value and the third output.

In at least one example embodiment, the training the AI model further includes continuously adjusting parameters of the plurality of machine learning models until the first output equals the first true value, the second output equals the second true value, and the third output equals the third true value.

In at least one example embodiment, the first true value is a calculated IPA morphology metric value, the second true value is a calculated sVRI morphology metric value, the third true value is a calculated SQI morphology metric value, the first output is an estimated IPA morphology metric, the second output is an estimated sVRI morphology metric, and the third output is an estimated SQI morphology metric.

In at least one example embodiment, the program of instructions includes instructions to cause the processor to re-train the AI model.

In at least one example embodiment, each machine learning model of the plurality of machine learning models is configured to respectively determine one of an IPA morphology metric of the PPG data, a sVRI morphology metric of the PPG data, or a SQI morphology metric of the PPG data.

In at least one example embodiment, a method is provided, the method including providing, as an input, Photoplethysmography (PPG) data to an AI model, the AI model including a plurality of machine learning models and the PPG data including PPG signals obtained from an individual, processing, by the plurality of machine learning models, the PPG data to determine one or more morphology metrics, and outputting, by the AI model, a statistical value based on the one or more morphology metrics.

In at least one example embodiment, the method further includes linking, by the AI model, the statistical value to one or more health conditions of the individual based on the one or more morphology metrics, wherein the one or more morphology metrics include, an inflection point area ratio (IPA) morphology metric of the PPG data, a stress-induced vascular response index (sVRI) morphology metric of the PPG data, and a signal quality index (SQI) morphology metric of the PPG data.

In at least one example embodiment, at least some of the processes described herein may be carried out by an apparatus comprising means for carrying out at least some of the described processes. In at least one example embodiment, a processing device comprising means for carrying out a method of training an AI model to process PPG data may be provided.

Further areas of applicability will become apparent from the description provided herein. The description and specific examples in this summary are intended for purposes of illustration only and are not intended to limit the scope of the present inventive concepts.

### BRIEF DESCRIPTION OF THE DRAWINGS

Example embodiments will become more fully understood from the detailed description given herein below and the accompanying drawings, wherein like elements are represented by like reference numerals, which are given by way of illustration only and thus are not limited to the example embodiments.

FIG. 1 illustrates an overview of a method of training an artificial intelligence (AI) model to process PPG signals, according to some example embodiments.

FIG. 2 illustrates an AI model, according to some example embodiments.

FIG. 3 illustrates a method of training an AI model, according to some example embodiments.

FIG. 4 illustrates training operations included in the method of training an AI model of FIG. 3.

FIG. 5 illustrates a block diagram of an apparatus, according to some example embodiments.

The various features and advantages of the non-limiting example embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

### DETAILED DESCRIPTION

Various example embodiments will now be described more fully with reference to the accompanying drawings in which some example embodiments are shown.

Detailed illustrative embodiments are disclosed herein. However, specific structural and functional details disclosed herein are merely representative for purposes of describing example embodiments. The example embodiments may, however, be embodied in many alternate forms and should not be construed as limited to only the embodiments set forth herein.

It should be understood that there is no intent to limit example embodiments to the particular forms disclosed. On the contrary, example embodiments are to cover all modifications, equivalents, and alternatives falling within the scope of this disclosure. Like numbers refer to like elements throughout the description of the figures.

The terminology used herein is for the purposes of describing the various example embodiments only and is not intended to be limiting of the various example embodiments. As used herein, the singular forms “a”, “an”, and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, and/or elements, but do not preclude the presence or addition of one or more other features, integers, steps, operations, elements, and/or groups thereof.

The following embodiments are examples. Although the specification may refer to “an”, “one”, or “some” example embodiment(s) in several locations of the text, this does not necessarily mean that each reference is made to the same embodiment(s), or that a particular feature only applies to a single example embodiment. Single features or different example embodiments may also be combined to provide other example embodiments. Further, when a particular feature, structure, or characteristic is described in connection of an example embodiment, it is within the knowledge of one skilled in the art to apply such feature, structure, or characteristic in connection with other example embodiments, whether or not explicitly described. It shall be understood that although the terms “first,” “second,” and the like may be used herein to describe various elements, these elements should not be limited by these terms. These terms are only used to distinguish one element from another.

Specific details are provided in the following description to provide a thorough understanding of the example embodiments. However, it will be understood by one of ordinary skill in the art that example embodiments may be practiced without these specific details. For example, systems may be shown in block diagrams in order not to obscure the example embodiments in unnecessary detail. In other instances, well-known processes, structures, and techniques may be shown without unnecessary detail in order to avoid obscuring example embodiments.

Also, it is noted that example embodiments may be described as a process depicted as a flowchart, a flow diagram, a data flow diagram, a structure diagram, or a block diagram. Although a flowchart may describe the operations as a sequential process, many of the operations may be performed in parallel, concurrently or simultaneously. In addition, the order of operations may be re-arranged. A process may be terminated when its operations are completed, but may also have additional steps not included in the figure. A process may correspond to a method, a function, a procedure, a subroutine, a subprogram, etc. When a process corresponds to a function, its termination may correspond to a return of the function to the calling function or the main function.

While one or more example embodiments may be described from the perspective of a function, it should be understood that one or more example embodiments discussed herein may be performed by one or more processors (or processing circuitry) at the applicable device, apparatus, and/or system. For example, according to one or more example embodiments, at least one memory may store instructions that, when executed by one or more processors, cause the device, the apparatus, system, module, or the like, to perform the operations discussed herein.

It will be appreciated that a number of example embodiments described herein may be used in combination. Features from one or more of the example embodiments may be selected to create alternate implementations comprised of sub-combination of features which may not be explicitly described above. In addition, features from one or more of the example embodiments may be selected and combined to create alternate implementations comprised of a combination of features which may not be explicitly described. Features suitable for such combinations and sub-combinations would be readily apparent to persons of ordinary skill in the art.

When the term “substantially” or “approximately” is used in this specification in connection with a numerical value, it is intended that the associated numerical value includes a manufacturing or operational tolerance (e.g., ±10%) around the stated numerical value. Moreover, when the term “substantially” is used in connection with geometric shapes, it is intended that precision of the geometric shape is not required but that latitude for the shape is within the scope of the disclosure. Further, regardless of whether numerical values or shapes are modified as “substantially,” it will be understood that these values and shapes should be construed as including a manufacturing or operational tolerance (e.g., ±10%) around the stated numerical values or shapes.

It will be understood that elements and/or properties thereof may be recited herein as being “the same” or “equal” as other elements, and it will be further understood that elements and/or properties thereof recited herein as being “identical” to, “the same” as, or “equal” to other elements may be “identical” to, “the same” as, or “equal” to the other elements and/or properties thereof. Elements and/or properties thereof that are “the same” or “equal” to other elements and/or properties thereof will be understood to include elements and/or properties thereof that are identical to, the same as, or equal to the other elements and/or properties thereof within manufacturing tolerances and/or material tolerances (e.g., ±10%). Elements and/or properties thereof that are identical, the same, and/or equal as other elements and/or properties thereof may be structurally the same or substantially the same, functionally the same or substantially the same, and/or compositionally the same or substantially the thereof.

Various example embodiments are generally related to processing photoplethysmography (PPG), and more particularly, to methods of using and training an artificial intelligence (AI) model to process PPG data including PPG signals.

Currently, PPG research and development is characterized by a lack of pre-trained models that can be readily adapted for various tasks. Unlike the rapid advancements in natural language processing and computer vision, where foundation models (FMs) have revolutionized different fields by providing powerful, general-purpose architectures that can be fine-tuned for a wide range of downstream applications, the PPG domain has not yet seen the emergence of such foundational models. Some systems related to PPG signals focus on single datasets and narrow, task-specific models. This is despite the fact that a single PPG signal stream can be used to infer a diverse set of bio-signals and health indicators, including heart rate variability (HRV), blood oxygen saturation (SpO2), respiration rate (RR), blood pressure (BP), arterial stiffness, as well as higher-level assessments of stress, sleep quality, emotion, and physical activity. The absence of a generalizable, pre-trained foundation models (FMs) capable of capturing these representations has hindered progress and limited the scope of research and innovation in the PPG domain.

Some efforts to create comprehensive PPG datasets may have been hindered by technical challenges, such as obtaining large-scale high-quality datasets. There has been a steady increase in PPG datasets collected using heterogeneous devices, sampling rates, and populations, which enables the potential for PPG foundation model (FM) development and evaluation. However, no existing effort has systematically curated PPG datasets for building robust, generalizable models (e.g., foundation models (FMs)). Traditional pre-training methods, such as contrastive learning, may not be well-suited to capture unique high-dimensional time series and PPG features. Accordingly, the physiological signal processing community currently lacks a pre-trained model comparable to FMs in other fields.

Some systems may include self-supervised learning (SSL). In some systems, SSL may be used for learning general representations from unlabeled datasets, with applications in physiological signal analysis including health records, fitness, brain and heart signals. Despite SSL's popularity, there are no widely used pre-trained models for PPG data. Some studies have demonstrated that embeddings from electrocardiogram (ECG) signals and PPG signals can be generalized across multiple health outcomes using data obtained by wearable devices such as smartwatches. Some studies have shown that embedding PPG signals can improve genetic discovery and risk prediction outcomes using datasets (e.g., such as the UK Biobank dataset). Some studies have also explored PPG embeddings for various applications. However, these studies often used single-device datasets, did not explore out-of-domain generalization, and/or did not release their models, which may highlight the need for openly available, pre-trained PPG foundation models (FMs).

Some systems may include generic time series FMs, which have recently gained popularity. However, these FMs often lack significant physiology data representation. While knowledge from generic models may be transferred to PPG tasks, performance is likely to be limited compared to a PPG-specific model. Similarly, transferring from other domain-specific models (e.g., electrocardiogram (ECG) and/or electroencephalogram (EEG)) to PPG may be challenging due to differences in signal characteristics. Some systems have implemented models using PPG signals from a single subject, however, said systems do not train and evaluate a FM using publicly available PPG datasets from a plurality of subjects.

Some example embodiments provide an AI model including a set of pre-trained models (also referred to as PAPAGEI) that may serve as a backbone (also referred to as a base or foundation) for a variety of PPG-related tasks. The PAPAGEI may be pre-trained based on a large-scale pre-training that enables the set of pre-trained models to capture rich and diverse PPG representations through large-scale pre-training.

In some example embodiments, the PAPAGEI may be pre-trained based on a large-scale pre-training for PPG signals. The PAPAGEI may be an open FM pre-trained on PPG signals, using about 57,000 hours of PPG datasets from about 20 million signals sourced from publicly available data sources. The PAPAGEI may be the first open FM pre-trained on PPG signals using the aforementioned PPG datasets, which may represent a new benchmark for large-scale model development in the domain of wearable and clinical signals (or clinical health monitoring).

In some example embodiments, the PAPAGEI may include a PPG-aware self-supervised learning (SSL) framework for PPG data, including a PPG signal morphology augmentation module. In some example embodiments, the PAPAGEI may be configured to use self-supervised learning (SSL) to train a deep neural network encoder included in the PAPAGEI. In some example embodiments, the PAPAGEI may be configured to operate in a first mode or a second mode. The first mode may be a patient contrastive SSL mode configured to maximize agreement between PPG signals from the same subject. The second mode may be a morphology-aware SSL mode that maximizes agreement between PPG signals that exhibit similar morphology across participants (or individuals, or subjects).

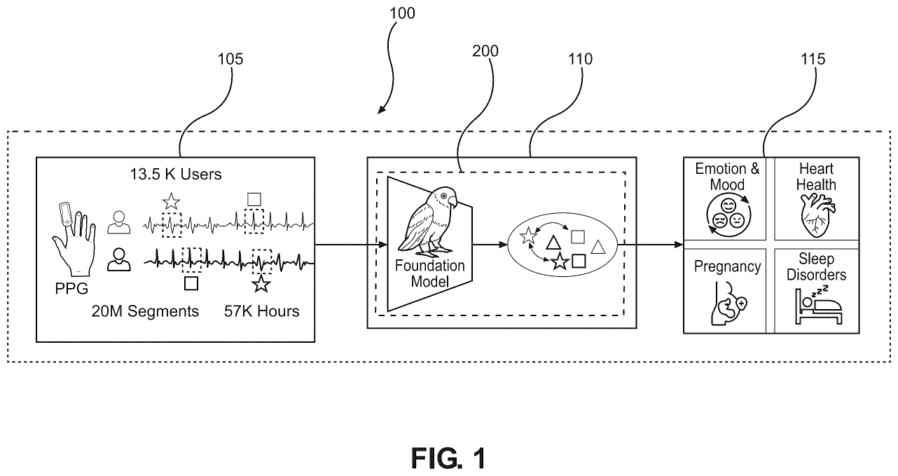

FIG. 1 illustrates an overview of a method 100 of training an artificial intelligence (AI) model to process PPG signals, according to some example embodiments.

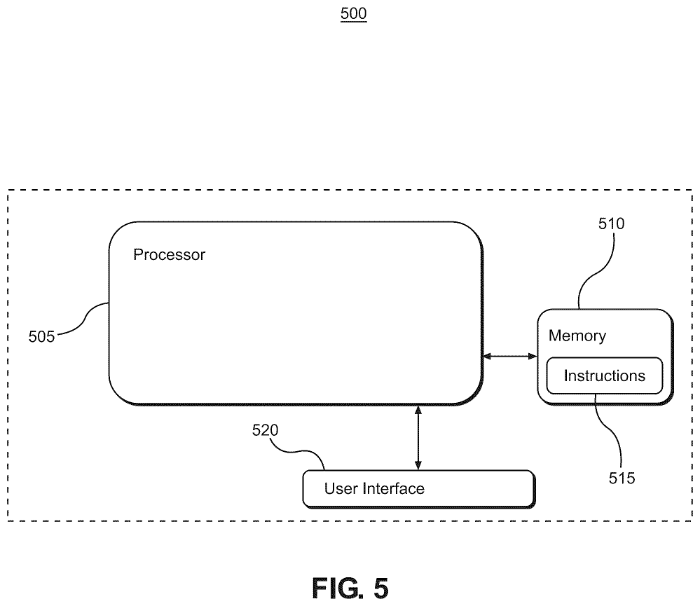

Referring to FIG. 1, at operation 105 a processing device (described later with regard to FIG. 5, for example) obtains one or more PPG datasets (also referred to herein as PPG data). A PPG dataset includes PPG signals from S number of individuals (or subjects or users). Each PPG signal may include one or more segments of a waveform.

In more detail, for example, a PPG dataset is given by D={p¹, p², . . . p^{S}}, where p^{s} is the PPG signal. The PPG signal p^{s}∈ [symbol] ⁿ may be defined as a time series that models changes in light intensity due to arterial blood flow. To model granular changes in a PPG signal obtained from subject S, PPG signals p^{S} are segmented without overlap to obtain X^{S}={x^{S}₁, x^{S}₂, . . . x^{S}_{N}}, where N is the number of fixed-length waveform segments obtained after segmenting a PPG signal.

In some example embodiments, the PPG datasets may include approximately 57,000 hours of data from about 20 million PPG signals sourced from publicly available data sources, but example embodiments are not limited thereto. For example, the PPG datasets may include more or less than approximately 57,000 hours from more or less than approximately 20 million PPG signals from public data sources and/or private data sources.

The PPG signals included in the PPG datasets may be obtained by wearable devices such as smartwatches and/or any other device suitable to detect and/or sense PPG signals from an individual. The PPG datasets may include PPG signals from a plurality of individuals. The PPG signals may each have one or more segments of one or more waveforms.

Still referring to FIG. 1, at operation 110, the processing device processes the PPG datasets to generate and output an output data. In one example, the processing device uses an AI model 200 to process the PPG datasets. The AI model 200 may be implemented by a processor (e.g., as shown in FIG. 5) coupled to a memory (e.g., as shown in FIG. 5) included in the processing device. For example, the memory may be configured to store a program of instructions and the processor coupled to the memory may be configured to execute the program of instructions to implement the AI model 200. The processing device may be and/or include a non-transitory computer-readable storage medium (e.g., the memory). The processing device will be described in more detail further below with reference to FIG. 5

The AI model 200 may be configured to use self-supervised learning (SSL) to train a deep neural network encoder (or a neural network encoder) included in the AI model 200. The AI model 200 may be trained based on a first training method and/or a second training method. In both the first training and the second training, the AI model 200 is configured to process PPG datasets to generate output data, and to output the output data.

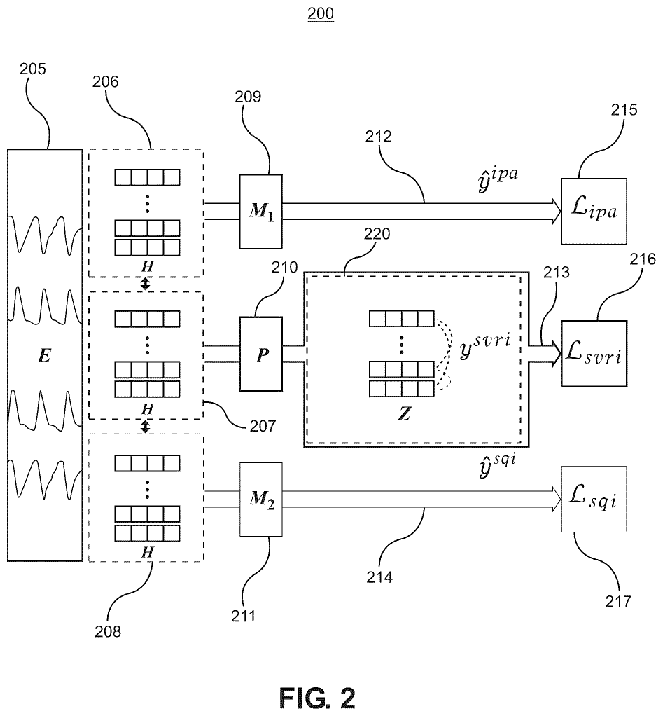

In some example embodiments, the AI model 200 may include a plurality of pre-trained models (also referred to as a plurality of pre-trained machine learning models). The plurality of pre-trained models may include a first machine learning model, a second machine learning model, and a third machine learning model, but example embodiments are not limited thereto. For example, the AI model 200 may include one or more machine learning models. The first machine learning model may include a first neural network, the second machine learning model may include a second neural network, and the third machine learning model may include a third neural network. The AI model 200 will be described in more detail below with reference to FIG. 2.

Still referring to FIG. 1, at operation 115, the processing device links the output data from the AI model 200 to one or more health conditions. To link the output data, the processing device evaluates the output data to determine whether the PPG signals in the processed PPG datasets may be linked to one or more health conditions. As shown in FIG. 1, the one or more health conditions may relate to emotion and mood, heart health, pregnancy, and/or sleep disorders, but example embodiments are not limited thereto. An example embodiment of a method of linking the output data will be described in more detail below.

FIG. 2 illustrates an AI model, according to some example embodiments. The AI model 200 in FIG. 2 corresponds to the AI model 200 described above with reference to FIG. 1.

Referring to FIG. 2, the AI model 200 includes an encoder 205 and a plurality of machine learning models including a first machine learning model 209, a second machine learning model 210, and a third machine learning model 211. Although described with regard to three machine learning models, the AI model 200 may include more or less than three machine learning models.

The first machine learning model 209 includes a first neural network, the second machine learning model 210 includes a second neural network, and the third machine learning model 211 includes a third neural network.

In the example embodiment shown in FIG. 2, the encoder 205 receives training data including PPG datasets including PPG signals. The PPG datasets may be obtained during operation 105 described above with reference to FIG. 1.

The encoder 205 processes the PPG datasets to determine a plurality of embeddings of the PPG signals included in the PPG datasets. The plurality of embeddings include a first embedding 206 of PPG signals corresponding to the first machine learning model 209, a second embedding 207 of PPG signals corresponding to the second machine learning model 210, and a third embedding 208 of PPG signals corresponding to the third machine learning model 211. The encoder 205 may provide the first embedding 206 to the first machine learning model 209, the second embedding 207 to the second machine learning model 210, and the third embedding 208 to the third machine learning model 211. The first embedding 206, the second embedding 207, and the third embedding 208 include a first data H (or a first set of data H). In this regard, the first embedding 206, the second embedding 207 and the third embedding 208 may be the same such that the same embeddings are provided to each of the first machine learning model 209, the second machine learning model 210 and the third machine learning model 211.

Based on the respective embeddings, the first machine learning model 209, the second machine learning model 210, and the third machine learning model 211 are configured to generate respective loss functions. In at least one example embodiment, the first machine learning model 209 may generate a first output 212 and then a first loss function 215 based on a first true value and the first output 212. The second machine learning model 210 may generate a second output 213 and then a second loss function 216 based on a second true value and the second output 213. The second output 213 may include a fourth embedding 220, which may include a second data Z (or a second set of data Z). In some example embodiments, the second data Z may be different from the first data H. The third machine learning model 211 may generate a third output 214 and then a third loss function 217 based on a third true value and the third output 214.

In some example embodiments, the first output 212 is an estimated inflection point area ratio (IPA) morphology metric related to the PPG signals in the PPG datasets. The second output 213 is an estimated stress-induced vascular response index (sVRI) morphology metric related to the PPG signals in the PPG datasets. The third output 214 is an estimated signal quality index (SQI) morphology metric related to the PPG signals in the PPG datasets.

The first true value may be related to a calculated IPA morphology metric value corresponding to the PPG datasets, the second true value may be related to a calculated sVRI morphology metric value corresponding to the PPG datasets, and the third true value may be related to a calculated SQI morphology metric value corresponding to the PPG datasets.

The first loss function 215, the second loss function 216 and the third loss function 217 are determined based on a difference between the estimated morphology metrics corresponding to the PPG datasets (e.g., the first output 212, the second output 213 and the third output 214) and the calculated morphology metrics (e.g., related the first to third true values). The AI model 200 may be configured to continuously adjust parameters of the plurality of machine learning models until the first output 212 is equal or substantially equal to the first true value, the second output 213 is equal or substantially equal to the second true value, and the third output 214 is equal or substantially equal to the third true value. In some example embodiments, example methods for calculating the first true value, the second true value and the third true value by the AI model 200 are discussed below. In some example embodiments, the AI model 200 may be configured to re-train the plurality of machine learning models based on one or more particular values (or desired values) of the first to third loss functions 215-217, particular PPG datasets being input to the AI model 200, a time elapsed since most recent training (e.g., a periodical training), and/or updates in the set of instructions stored in the memory of the processing device that implements the AI model 200, but example embodiments are not limited thereto.

Noise in the PPG datasets may be filtered before processing the PPG datasets to generate the first embedding 206, the second embedding 207 and the third embedding 208 and/or before the PPG datasets are input to the encoder 205. The AI model 200 may be configured to link the PPG datasets input to the encoder 205 to one or more health conditions based on processing of the PPG datasets. For example, the AI model 200 may be configured to link one or more of the first to third outputs 212-214 to the one or more health conditions based on comparing the first output 212, the second output 213 and the third output 214 to morphology metrics related to particular health conditions. In some example embodiments, the AI model 200 is configured to generate and output a command (e.g., a command signal) to an external device (e.g., a monitor, a user interface, a controller, etc.) to display the linked health conditions associated with the first loss function 215, the second loss function 216, and the third loss function 217. In some example embodiments, the AI model 200 is configured to generate and output a report to a user including the first to third loss functions 215-217 and the linked health conditions associated with the first to third loss functions 215-217.

The calculated morphology metric values (e.g., IPA, sVRI, and SQI) of the first to third true values and the estimated morphology metrics of the first output 212, the second output 213 and the third output 214 may correspond to the PPG datasets. For example, the calculated morphology metric values and the estimated morphology metrics may be related to one or more segments of the PPG signals in the PPG datasets that are input to the encoder 205.

The one or more segments of PPG signals are related to at least one segment of a waveform of a PPG signal obtained from an individual. The PPG signal may have been acquired using a wearable device (e.g., a smartwatch), but example embodiments are not limited thereto. For example, the PPG signal corresponding to the one or more segments may have been acquired (or sensed, or detected) using a smartwatch or any other device configured to acquire PPG signals from the individual.

As noted above, the plurality of machine learning models included in the AI model 200 may be trained according to a first training and/or a second training.

The first training may also be referred to as a participant-aware objective training. The first training may be a patient contrastive SSL training (also referred to as PAPAGEI-P) configured to maximize agreement between PPG signals from the same subject.

According to the first training, the plurality of machine learning models may be configured to define a positive pair as any two distinct segments (e.g., of PPG signals) from the same subject, denoted as

$$\{ ( {x}_{i}^{s} , {x}_{j}^{s} ) \vert i \neq j \} ,$$

and apply a series of random time series augmentation to obtain

$$( {\tilde{x}}_{i}^{s} , {\tilde{x}}_{j}^{s} ) .$$

In some example embodiments, augmentations such as random cropping, adding Gaussian noise, time flipping, negation, and magnitude scaling may be used, but example embodiments are not limited thereto. Each augmentation may be applied based on a given, desired or predefined probability, which may determine whether it will be used (or whether it may be beneficial to use the first training). Additionally or alternatively, each augmentation may include hyper-parameters that may control the intensity of the transformation.

In some example embodiments, during training of the AI model 200 based on the first training, two randomly sampled positive pairs may be passed through the encoder 205 (also referred to in FIG. 2 as the encoder E) and the second machine learning model 210 (also referred to in FIG. 2 as the Projection P) to obtain the fourth embedding 220 denoted

$$( {z}_{i}^{s} , {z}_{j}^{s} ) .$$

Given a batch of fourth embeddings from N distinct subjects with positive pairs of the form

$$( {z}_{i}^{s} , {z}_{j}^{s} ) ,$$

the AI model 200 may compute and/or optimize the normalized temperature-scaled cross entropy (NT-Xent) loss [symbol] ₚ provided by Equation 1 below.

$${ℒ}_{p} = \frac{1}{2} ( {ℓ}_{p} ( i , j ) + {ℓ}_{p} ( j , i ) ) , where \qquad [ Equation 1 ]$$

$${ℓ}_{p} ( i , j ) = - \frac{1}{N} {\sum }_{u = 1}^{N} log \frac{exp ( sim ( {z}_{i}^{u} , {z}_{j}^{u} ) / τ )}{{\sum }_{υ = 1}^{2 N} 𝟙 [ υ \neq u ] exp ( sim ( {z}_{i}^{u} , {z}_{u}^{υ} ) / τ )}$$

In Equation 1, lₚ is a loss function (e.g., [symbol] ₚ(i,j)) and sim represents any similarity function or measure including two vectors and its calculated similarities (e.g., the calculated similarities of the two vectors). For example, sim may refer to a cosine similarity function of two vectors.

The second training may be referred to as a segment-aware objective training. The second training may be a morphology-aware SSL training (also referred to as PAPAGEI-S) configured to maximize agreement between PPG signals that exhibit similar morphology across participants.

In PPG signals, variations caused by a total peripheral resistance (TPR) (e.g., the force exerted by the body's blood vessels on circulating blood), are reflected, which may affect distinct regions within the waveform of the PPG signals. TPR varies under certain medical conditions, such as hypertension and diabetes; thus, fluctuations in TPR may be used (or key, or crucial) for identifying adverse medical conditions. To incorporate morphology into SSL, a morphology augmentation module may be introduced prior to training. The morphology augmentation module may be configured to calculate (or compute, or determine) three PPG morphology metrics. The PPG morphology metrics may include: (1) the sVRI, which may be the ratio of mean PPG signal between post to pre-systolic phases; (2) the IPA, which may be the ration of systolic to diastolic areas defined by the dicrotic notch; and (3) the SQI, which may be a skewness of the signal as an indicator of quality of the signal.

The three PPG morphology metrics complement each other. For example, sVRI captures amplitude variations of the signals, IPA reflects signal width, and SQI addresses cases where IPA cannot be computed due to poor-quality signals lacking a dicrotic notch. In one example, the PPG morphology metric sVRI may be calculated according to Equation 2 shown below, the PPG morphology metric IPA may be calculated according to Equation 3 shown below, and the PPG morphology metric SQI may be calculated according to Equation 4 shown below.

$$sVRI ( x ) = \frac{sys {\sum }_{i = sys}^{n} {x}_{i}}{( n - sys ) {\sum }_{i = 1}^{sys} {x}_{i}} \qquad [ Equation 2 ]$$

$$IPA ( x ) = \frac{{\int }_{0}^{\hat{n}} \times dn}{{\int }_{\hat{n}}^{n} \times dn} \qquad [ Equation 3 ]$$

$$SQI ( x ) = \frac{1}{W} {\sum }_{w} \frac{{m}_{3}}{{m}_{2}^{3 / 2}} \qquad [ Equation 4 ]$$

In Equations 2-4, (x∈ [symbol] ⁿ) is the PPG segment, sys is the systolic peak, n is the length of time series, W is the total windows, i is an index for counting, xᵢ is the data representing the PPG signals (e.g., data representing PPG signals in PPG datasets), and n is the dicrotic notch. For SQI, x is divided into 5 second windows (each window is w), and the skewness mᵢ is computed according to Equation 5 shown below.

$${m}_{i} = \frac{1}{5 \times f} {\sum }_{j = 1}^{5 \times f} {( x [ j ] - {μ}_{x} [ j ] )}^{i} \qquad [ Equation 5 ]$$

In Equation 5, x is the data representing the PPG signals (e.g., data representing PPG signals in PPG datasets), μₓ is the mean value of x, and f is the sampling rate frequency for the PPG signal.

In some example embodiments, the window size provided by Equations 1-5, may provide the best (or an optimal or improved) signal quality discrimination.

The one or more segments may be segments of a waveform of a PPG signal corresponding to a cardiac cycle of an individual represented by the PPG signals included in the PPG datasets, but example embodiments are not limited thereto. The one or more segments may include a first portion, a second portion, a first area, a second area, a peak, and a notch. The first portion may correspond to a pre-systolic phase of a cardiac cycle of an individual, the second portion may correspond to a post-systolic phase of a cardiac cycle of an individual, the first area may be a systolic phase area of a cardiac cycle of an individual, the second area may be diastolic phase area of a cardiac cycle of an individual, the peak may be a systolic peak of a cardiac cycle of an individual, and the notch may be a dicrotic notch of a cardiac cycle of an individual.

The second training further includes performing a morphology augmentation that includes an augmented (e.g., only Gaussian noise or cropping) input time series (x) and outputs y={y^{svri}, y^{ipa}, y^{sqi}}∈ [symbol] ³, where y^{svri}, y^{ipa}, and y^{sqi} are associated with the first output 212, the second output 213, and the third output 214, respectively. In some example embodiments, to denote positive pairs, the training may include discretizing y^{svri} into a set (e.g., a predefined set) of n=8 bins, where y^{svri}∈{0,1, . . . , n}. The second training may also include defining positive pairs based on sVRI labels as

$$\{ ( {x}_{i} , {x}_{j} \} ) \vert {y}_{i}^{svri} = {y}_{j}^{svri} , i \neq j \} ,$$

where positive pairs are not defined on subjects. In some example embodiments, the second training includes optimizing three heads (e.g., the plurality of machine learning models including the first to third machine learning models 209-211) based on a batch of N PPG signals and their respective morphology. The fourth embedding 220 Z={z₁, z₂, . . . , z_{N}} from the projection P (or from the second machine learning model 210) may be extracted, and the contrastive loss for sVRI (the second loss function 216) may be computed using, for example, Equations 6 and 7 shown below.

$${ℓ}_{s} ( i , j ) = - log \frac{exp ( sim ( {z}_{i} , {z}_{j} ) / τ )}{{\sum }_{k = 1}^{2 N} 𝟙 [ k \neq i ] exp ( sim ( {z}_{i} , {z}_{k} ) / τ )} \qquad [ Equation 6 ]$$

In Equation 6, lₛ represents a loss function, k is an index, and T is a hyperparameter (e.g., temperature) that may be adjusted based on the desired application. The function sim is a similarity function as discussed above.

$${ℒ}_{s υ ri} = \frac{1}{2 N} {\sum }_{k = 1}^{N} [ ℓ ( 2 k - 1 , 2 k ) + ℓ ( 2 k , 2 k - 1 ) ] \qquad [ Equation 7 ]$$

The first machine learning model 209 may predict the IPA (ŷ^{ipa}∈ [symbol] ^{N}) as the first output 212 and the second machine learning model 211 may predict the SQI (ŷ^{sqi}∈ [symbol] ^{N}) as the third output 214 based on the respective embeddings 206 and 208 (e.g., H={h₁, h₂, . . . , h_{N}} including the first data H). The first machine learning model 209 may be a mixture of expert (MoE) head M₁ and the second machine learning model 211 may be a MOE head M₂. Each MoE head may include three fully connected neural networks (FCNNs), but example embodiments are not limited thereto. For example, the first machine learning model 209 and the third machine learning model 211) may include more or less FCNNs and/or may include one or more different types of neural networks. In one example, the first output 212 and the third output 214 may be calculated as a weighted sum of the FCNNs based on using softmax to determine the respective weights, but example embodiments are not limited thereto. The first machine learning model 209 and the third machine learning model 211 may be optimized based on the loss functions, which are based on the mean absolute error, as shown below in Equations 8 and 9.

$${ℒ}_{ipa} = \frac{1}{N} {\sum }_{i = 1}^{N} \vert {y}_{i}^{ipa} - {\hat{y}}_{i}^{ipa} \vert \qquad [ Equation 8 ]$$

$${ℒ}_{sqi} = \frac{1}{N} {\sum }_{i = 1}^{N} \vert {y}_{i}^{sqi} - {\hat{y}}_{i}^{sqi} \vert \qquad [ Equation 9 ]$$

In some example embodiments, morphology indices may encapsulate various PPG characteristics. Each of the first machine learning model 209 and the third machine learning model 211 may be trained to specialize in learning distinct properties that may contribute to an overall index of the AI model 200. In some example embodiments, the overall training objective of the second training may be provided by Equation 10 below.

$${ℒ}_{s} = α {ℒ}_{s υ ri} + ( 1 - α ) ( {ℒ}_{ipa} + {ℒ}_{sqi} ) , where α ∈ [ 0 , 1 ] \qquad [ Equation 10 ]$$

In Equation 10, a is a value between 0 and 1.

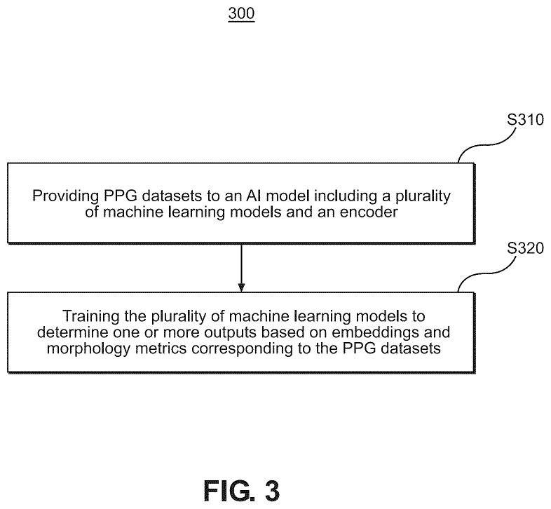

FIG. 3 illustrates a method of training an AI model, according to some example embodiments. The method 300 may be a method of training the AI model 200 described above with reference to FIG. 2.

Referring to FIG. 3, at operation S310, the AI model 200 receives training data including, for example, PPG datasets including PPG signals. As described above with reference to FIGS. 1-2, the AI model 200 may include the encoder 205, the first machine learning model 209, the second machine learning model 210, and the third machine learning model 211. The encoder 205 may be configured to receive and process the PPG datasets to generate embeddings corresponding to the PPG signals in the PPG datasets. The encoder 205 outputs the embeddings to the plurality of machine learning models 209-211.

At operation S320, the plurality of machine learning models 209-211 are trained based on the first training and/or the second training to determine one or more outputs based on the embeddings and morphology metrics corresponding to the PPG datasets included in the training data. In some example embodiments, the training the plurality of machine learning models 209-211 may include training the AI model 200, using the training data, to determine one or more loss functions [symbol] ᵢₚₐ, [symbol] ₛᵥᵣᵢ, and/or [symbol] _{sqi} as described above with regard to FIGS. 1 and 2. In some example embodiments, the training the AI model may include using the one or more loss functions to determine and/or adjust the one or more outputs. As described above with reference to FIG. 2, the AI model 200 may be configured to calculate morphology metrics including estimated values and true values of at least three morphology metrics corresponding to the PPG datasets. The at least three morphology metrics may be an IPA ratio morphology metric corresponding to the PPG datasets, a sVRI morphology metric corresponding to the PPG datasets, and a SQI morphology metric corresponding to the PPG datasets. As discussed above, the training the AI model 200 may include training the plurality of machine learning models to process embeddings of PPG signals and generate and output one or more outputs including estimated morphology metrics based on the embeddings of PPG signals. In some example embodiments, to generate the one or more outputs, the plurality of machine learning modules may be configured to calculate true values of the morphology metrics and determine loss functions based on a difference between the estimated morphology metrics and the calculated true values of the morphology metrics. In some example embodiments, the AI model 200 may be trained to continuously adjust parameters of the plurality of machine learning modules until the one or more outputs including the estimated morphology metrics are equal or substantially equal to the calculated true values of the morphology metrics.

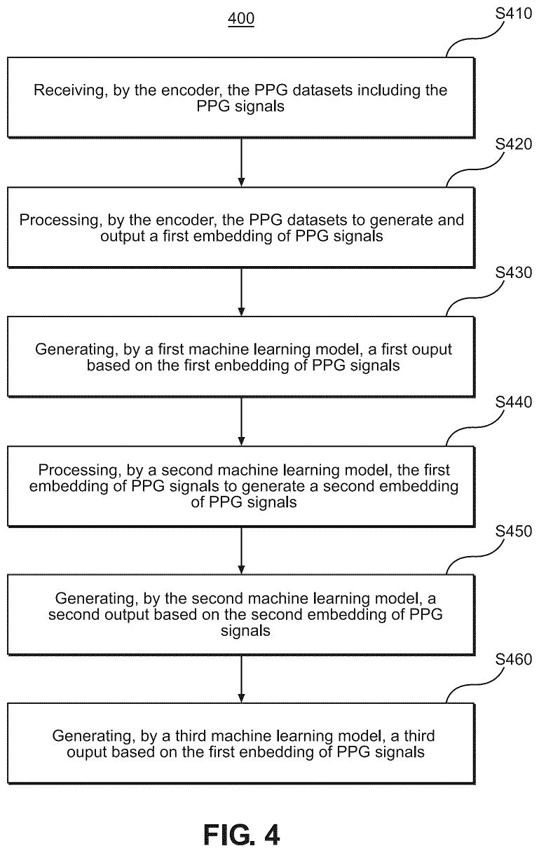

FIG. 4 illustrates an example embodiment of a method 400 of training an AI model at operation S320 in FIG. 3. For example purposes, the example embodiment shown in FIG. 4 will be discussed with regard to the example embodiment shown in FIG. 2.

Referring to FIG. 4, at operation S410, the encoder 205 receives PPG datasets including PPG signals.

At operation S420, the encoder 205 processes the PPG data to generate and/or extract embeddings 206-208 of PPG signals and output the embeddings 206-208 of PPG signals to the plurality of machine learning models 209-211. As noted above, the embeddings 206-208 may be the same and include the same data/information such that the same or substantially the same information is provided to each of the plurality of machine learning models 209-211.

At operation S430, the first machine learning model 209 receives the embedding 206 of PPG signals and generates the first output 212 based on the embedding 206 of PPG signals.

At operation S440, the second machine learning model 210 receives and processes the embedding 207 of PPG signals to generate a fourth embedding 220 of PPG signals based on the embedding 207 of PPG signals.

At operation S450, the second machine learning model 210 generates the second output 213 based on the fourth embedding 220 of PPG signals.

At operation S460, the third machine learning model 211 receives the embedding 208 of PPG signals and generates the third output 214 based on the embedding 208 of PPG signals.

Additionally or alternatively, a method of using the AI model 200 may be provided. The method of using the AI model 200 may include providing PPG signals and/or PPG datasets to the AI model 200 by an individual. The PPG signals and/or PPG datasets may include PPG signals of the individual, but example embodiments are not limited thereto. The AI model 200 may be configured to process the PPG signals and/or PPG datasets based on the first training and/or the second training to determine one or more morphology metrics and output one or more statistical values based on the one or more morphology metrics. The AI model 200 may be configured to link the one or more statistical values to one or more health conditions of the individual (or associated to the PPG signals and/or PPG datasets provided by the individual) based on the one more morphology metrics. The one or more morphology metrics may include an IPA of the PPG data, a sVRI of the PPG data, and/or a SQI of the PPG signals and/or PPG datasets.

FIG. 5 illustrates a block diagram of an apparatus according to some example embodiments. The apparatus 500 may be a processing device configured to implement the AI model 200.

The apparatus 500 may be a processing device including at least one processor 505, a memory 510 configured to store instructions 515, and a user interface 520, but example embodiments are not limited thereto. For example, the processing device (or apparatus 500) may include more or less elements and/or parts. The at least one processor 505 may be configured to execute the instructions 515 stored in the memory 510 to cause the apparatus 500 to perform the methods of training and/or using the AI model 200 as disclosed herein. The memory 510 and the instructions 515 (e.g. a computer program code, software, etc.) are configured, with the at least one processor 505, to cause the apparatus 500 to perform the method or methods as disclosed herein, and any of the embodiments thereof.

The at least one processor 505 may comprise circuitry, or be constituted as circuitry and/or circuitries, the circuitry and/or circuitries being configured to perform phases (or operations) of methods in accordance with example embodiments described herein. As used in this application, the term “circuitry” (or circuitries) may refer to one or more or all of the following: (a) hardware-only circuit implementations, such as implementations in only analog and/or digital circuitry, and (b) combinations of hardware circuits and software, such as, as applicable: (i) a combination of analog and/or digital hardware circuit(s) with software/firmware and (ii) any portions of hardware processor(s) with software (including digital signal processor(s)), software, and memory(ies) that work together to cause an apparatus, such as a server, user equipment, or other electronic device, to perform various functions) and/or (c) hardware circuit(s) and/or processor(s), such as a microprocessor(s) or a portion of a microprocessor(s), that requires software (e.g., firmware) for operation, but the software may not be present when it is not needed for operation. This definition of circuitry (or circuitries) applies to all uses of this term in this application, including in any claims. As a further example, as used herein, the term circuitry (or circuitries) may also cover an implementation of merely a hardware circuit or processor (or multiple processors) or portion of a hardware circuit or processor and its (or their) accompanying software and/or firmware. The term circuitry (or circuitries) may also cover, for example, and if applicable to the particular claim element, a baseband integrated circuit or processor integrated circuit for a mobile device or a similar integrated circuit in server, a cellular network device, or other computing or network device.

The memory 510 may be implemented using any suitable data storage technology. The memory 510 may comprise a database for storing data. The memory 510 may be at least in part external to the apparatus 500 but accessible to the apparatus 500. The instructions 515 may be comprised in a computer readable medium or a non-transitory computer readable medium. A term non-transitory, as used herein, may be limitation of the medium itself (e.g., tangible, not a signal) as opposed to a limitation on data storage persistency (e.g., random access memory (RAM) vs. read only memory (ROM)).

The apparatus 500 may comprise a user interface 520 comprising, for example, at least one of a keypad, a microphone, a touch display, a display, a speaker, etc. The user interface 520 may be used to control the apparatus 500 by the user. The user interface 520 may be external to the apparatus 500. For example, the apparatus 500 may be connected to another device, such as a computer, either via wireless or wired connection, and the apparatus 500 may be controlled by the user via the computer.

In some example embodiments, three large public datasets may be used to pre-train the AI model 200. A first dataset may be VitalDB, which may include PPG signals collected during surgery at a sampling rate of 500 Hz from the finger of an individual using a patient monitor. A second dataset may be the MIMIC-III waveform database matched subset, where finger-tip PPG data may be collected at 125 Hz from an ICU (e.g., intensive care unit) monitor. A third dataset may be from the Multi-Ethnic Study of Atherosclerosis (MESA) sleep sub-study, which may provide PPG data obtained through finger-tip polysomnography at 265 Hz. During pre-training of the AI model 200, data from approximately 5,866 participants in the VitalDB dataset, approximately 2,055 participants in the MESA dataset, and approximately 5,596 participants in the MIMIC-III dataset may have been used. This corresponds to approximately 20,751,206 10-second segments, totaling approximately 57,641 hours of data.

In some example embodiments, to harmonize the PPG signals and extract information (or valuable information), the following steps may be performed: (1) a 4^{th}-order Chebyshev bandpass filter with low and high pass cutoffs set at 0.5 Hz and 12 Hz, respectively, may be applied to the PPG signals; (2) PPG signals may be segmented into 10-second windows; (3) flat line segments may be detected and segments where more than 25% of the data is flat may be removed; (4) the segments may be normalized using Z-score normalization; and (5) the segments may be resampled (or downsampled) to 125 Hz (which may be the lowest sampling rate in the pre-training datasets). In some example embodiments, the AI model 200 may be trained for approximately 15,000 steps on 8 V100 GPUs with a learning rate of approximately 10⁻⁴.

In some example embodiments, to evaluate the effectiveness of the AI model 200, the AI model 200 may be benchmarked against a range of diverse datasets, tasks, and baselines. The datasets used may be selected based on their size (or large sizes) and the clinical relevance of the tasks (where applicable). The tasks used may be approximately 20 tasks which may be based on clinical relevance. Table 1 below includes details on the tasks used.

TABLE 1

| | | SR | | | Task | #Subjects |
|---|---|---|---|---|---|---|
| #ID | Dataset | (Hz) | Collected by | Task | Type | (#Samples) |
| T1 | VitalDB | 500 | ICU monitor | ICU admission | B | 5866 |
| | | | | (Y/N) | | |
| T2 | VitalDB | | | Operation | M(11) | 5866 |
| | | | | Type | | |
| T3 | MIMIC-III | 125 | ICU Monitor | Mortality | B | 5596 |
| T4 | MESA | 256 | Polysomnography | Smoker | B | 2055 |
| | | | finger | | | |
| T5 | MESA | 256 | Polysomnography | AHI > 3% | R | 2055 |
| | | | finger | Oxygen Desat. | | |
| T6 | MESA | 256 | Polysomnography | AHI > 4% | R | 2055 |
| | | | finger | Oxygen Desat. | | |
| T7 | nuMom2B | 75 | Polysomnography | Pregnancy | B | 3163(5337) |
| | | | finger | stage | | |
| | | | | (early/late) | | |
| T8 | nuMom2B | 75 | Polysomnography | Gestation Age | B | 3163(5337) |
| | | | finger | | | |
| T9 | VV (Skin Tone) | 60 | Finger | Systolic BP | R | 231 |
| T10 | VV (Skin Tone) | 60 | Finger | Diastolic BP | R | 231 |
| T11 | PPG-BO | 1000 | Finger Pulse Ox | Systolic BP | R | 219 |
| T12 | PPG-BO | 1000 | Finger Pulse Ox | Diastolic BP | R | 219 |
| T13 | PPG-BO | 1000 | Finger Pulse Ox | Average Heart | R | 219 |
| | | | | Rate | | |
| T14 | PPG-BO | 1000 | Finger Pulse Ox | Hypertension | R | 219 |
| T15 | SDB | 62.5 | Finger Pulse Ox | Sleep Disorder | B | 146 |
| | | | | Breathing | | |
| T16 | ECSMP | 64 | Wrist | Mood | B | 89 |
| | | | | Disturbance | | |
| T17 | WESAD | 64 | Wrist | Valence | B | 15(4497) |
| T18 | WESAD | 64 | Wrist | Arousal | B | 15(4497) |
| T19 | PPG-DaLiA | 64 | Wrist | Heart Rate | R | 15(64697) |
| T20 | PPG-DaLiA | 64 | Wrist | Activity | R | 15(64697) |

Referring to Table 1 above, data in tasks 7-20 may be unseen during training of the AI model 200; thus, the corresponding tasks may be out-of-domain. The remaining tasks (e.g., tasks 1-6) may have been used for pre-training, but their test sets and labels may have been held out. For task type, B/M/R may refer to Binary classification, Multi-class classification (# classes), and Regression, respectively. Still referring to Table 1, systolic blood pressure, diastolic blood pressure, heart rate, hypertension, and activity may be related to cardiovascular health conditions; AHI>3% oxygen desaturation and AHI>4% oxygen desaturation may be related to sleep health conditions; valence, arousal, affect inducing videos, depression, and mood disturbance may be related to emotion and mood health conditions (or disorders); ICU admissions, operation type, mortality, and smoker may be related to hospital record; and pregnancy stage and gestion age may be related to obstetrics. Changes in gestational age and pregnancy stage may be risk factors associated with adverse pregnancy outcomes such as hypertensive disorders and small-for-gestational-age delivery. These diseases may affect heart function, which can be measured using a PPG sensor. Still referring to Table 1, tasks related to obstetrics may classify between early and late-stage pregnancy and may predict the gestational age of the fetus.

In some example embodiments, performance of the AI model 200 may be assessed by benchmarking such performance against several competitive baselines. As an open-source FM designed for physiological signal modeling, the AI model 200 may be compared to recent time-series FMs including Chronos Ansari and MOMENT, but example embodiments are not limited thereto. To evaluate the SSL framework of the AI model 200, the AI model 200 may be compared with modern SSL methos such as SimCLR, BYOL, and TF-C, but example embodiments are not limited thereto. Additionally or alternatively, to asses generalizability of PPG data of the AI model 200, the AI model 200 may be compared against REGLE, a published model pre-trained on UK Biobank's PPG signals. Additionally or alternatively, a random forest model may be incorporated as a task-specific baseline trained on signal features such as mean, median, maximum, minimum, and the 25^{th}, 50^{th}, and 75^{th} percentiles.

In some example embodiments, to perform a linear evaluation of the AI model 200, the ID and OOD datasets may be split into 80/10/10 and 60/20/20 training/validation/testing sets on the individual level, respectively, which may ensure no participant overlap between the different datasets. The AI model 200 may be analyzed by extracting feature representations and training probing models for each task. For binary classification task, a logistic regression model may be used, with performance assessed using the ROC-AUC. For regression tasks, a ridge regression may be used, and the performance may be evaluated using the mean absolute error (MAE). Multi-class classification tasks may be trained using a random model and may be evaluated based on accuracy. Additionally or alternatively, approximately 95% confidence intervals may be computed using bootstrapping (e.g., 500 sampling runs with replacement).

In at least one example embodiment, at least some of the processes described herein may be carried out by an apparatus (e.g., the apparatus 500) comprising means for carrying out at least some of the described processes. Means for performing method steps as disclosed herein may include software and/or hardware components of the apparatus 500. For example, at least one processor (e.g., the processor 505), a memory (e.g., the memory 510), and a computer program code (e.g., included in the instructions 515) may form means for carrying out the method or methods as disclosed herein, and any of the example embodiments thereof. As used herein the term “means” is to be construed in singular form, e.g., referring to a single element, or in plural form, e.g., referring to a combination of single elements. Therefore, terminology “means for [performing A, B, C]”, is to be interpreted to cover an apparatus in which there is only one means for performing A, B and C, or where there are separate means for performing A, B and C, or partially or fully over-lapping means for performing A, B, C. Further, terminology “means for performing A, means for performing B, means for performing C” is to be interpreted to cover an apparatus in which there is only one means for performing A, B and C, or where there are separate means for performing A, B, and C, or partially or fully overlapping means for performing A, B, C. At least one other example embodiment may include a computer program including program segments or instructions that, when executed by at least one processor of a system or an apparatus, cause the system or the apparatus to perform the method or methods disclosed herein.

Some example embodiments may include performing a comprehensive evaluation across diverse health-related tasks based on processing PPG signals using the AI model 200. The comprehensive evaluation may include a wide range of about 20 tasks, including cardiovascular health, sleep disorders, pregnancy monitoring, and overall well-being assessments.

Some example embodiments may include conducting extensive ablation studies based on processing PPG signals using the AI model 200. The extensive ablation studies may include conducting assessments of the impact of components (or key components) of the AI model 200 framework, conducting evaluations of the significance of signal morphology augmentation, comparing the framework of the AI model 200 with established contrastive learning approaches, and analyzing effects of different encoder architectures, model sizes, sampling rates, and augmentation techniques on the quality of PPG embeddings.

As discussed herein, the terminology “one or more” and “at least one” may be used interchangeably. Although the specification may refer to “an,” “one,” or “some” embodiment(s) in several locations of the text, this does not necessarily mean that each reference is made to the same example embodiment(s), or that a particular feature only applies to a single example embodiment. Single features of different example embodiments may also be combined to provide other example embodiments. Further, when a particular feature, structure, or characteristic is described in connection with an example embodiment, it is within the knowledge of one skilled in the art to apply such feature, structure, or characteristic in connection with other example embodiments whether or not explicitly described.

For the purposes of the present disclosure, the phrases “at least one of A or B,” “at least one of A and B,” and “A and/or B” means (A), (B), or (A and B). For the purposes of the present disclosure, the phrase “A, B, and/or C” means (A), (B), (C), (A and B), (A and C), (B and C), or (A, B, and C).

Although the terms first, second, etc., may be used herein to describe various elements, these elements should not be limited by these terms. These terms are only used to distinguish one element from another. For example, a first element could be termed a second element, and similarly, a second element could be termed a first element, without departing from the scope of this disclosure. As used herein, the term “and/or,” includes any and all combinations of one or more of the associated listed items. Similarly, the term “or” includes both its conjunctive and disjunctive meanings.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, figures shown in succession may in fact, be executed substantially concurrently or may sometimes be executed in the reverse order, depending upon the functionality/acts involved.

As discussed herein, illustrative example embodiments have been described with reference to acts and symbolic representations of operations (e.g., in the form of flow charts, flow diagrams, data flow diagrams, structure diagrams, block diagrams, etc.) that may be implemented as program modules or functional processes include routines, programs, objects, components, data structures, etc., that perform particular tasks or implement particular abstract data types and may be implemented using existing hardware at, for example, existing user equipment or other network elements and/or hardware. Such existing hardware may be processing or control circuitry such as, but not limited to, one or more processors, one or more Central Processing Units (CPUs), one or more controllers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUs), one or more microprocessors, one or more Application Specific Integrated Circuits (ASICs), or any other device or devices capable of responding to and executing instructions in a defined manner.

Although a flow chart may describe the operations as a sequential process, many of the operations may be performed in parallel, concurrently, or simultaneously. In addition, the order of the operations may be re-arranged. A process may be terminated when its operations are completed but may also have additional steps not included in the figure. A process may correspond to a method, function, procedure, subroutine, subprogram, etc. When a process corresponds to a function, its termination may correspond to a return of the function to the calling function or the main function.

As disclosed herein, the term “storage medium,” “computer-readable storage medium,” or “non-transitory computer-readable storage medium” may represent one or more devices for storing data, including read-only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other non-transitory, physical media capable of storing or instruction(s) and/or data.

Furthermore, example embodiments may be implemented by hardware, software, firmware, middleware, microcode, hardware description languages, or any combination thereof. When implemented in software, firmware, middleware, or microcode, the program code or code segments to perform the necessary tasks may be stored in a machine or computer-readable medium such as a computer-readable storage medium. When implemented in software, a processor or processors will perform the necessary tasks. For example, as mentioned above, according to one or more example embodiments, at least one memory may include or store computer program code, and the at least one memory and the computer program code may be configured to, with at least one processor, cause a network element or network device to perform the necessary tasks. Additionally, the processor, memory, and example algorithms, encoded as computer program code, serve as means for providing or causing performance of operations discussed herein.

A code segment of computer program code may represent a procedure, function, subprogram, program, routine, subroutine, module, software package, class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable technique including memory sharing, message passing, token passing, network transmission, etc.

The terms “including” and/or “having,” as used herein, are defined as comprising (i.e., open language). The term “coupled,” as used herein, is defined as connected, although not necessarily directly and not necessarily mechanically. Terminology derived from the word “indicating” (e.g., “indicates” and “indication”) is intended to encompass all the various techniques available for communicating or referencing the object/information being indicated. Some, but not all, examples of techniques available for communicating or referencing the object/information being indicated include the conveyance of the object/information being indicated, the conveyance of an identifier of the object/information being indicated, the conveyance of information used to generate the object/information being indicated, the conveyance of some part or portion of the object/information being indicated, the conveyance of some derivation of the object/information being indicated, and the conveyance of some symbol representing the object/information being indicated.

According to example embodiments, user equipment, other network elements, or the like may be (or include) hardware, firmware, hardware executing software, or any combination thereof. Such hardware may include processing or control circuitry such as, but not limited to, one or more processors, one or more CPUs, one or more controllers, one or more ALUs, one or more DSPs, one or more microcomputers, one or more FPGAs, one or more SoCs, one or more PLUs, one or more microprocessors, one or more ASICs, one or more quantum hardware circuits or any other device or devices capable of responding to and executing instructions in a defined manner.

Illustrative Embodiment 1. A method of training an artificial intelligence (AI) model, the method comprising providing training data to the AI model, the AI model including a plurality of machine learning models and the training data including Photoplethysmography (PPG) data based on PPG signals obtained from a plurality of individuals, and training the AI model, using the training data, to determine one or more outputs based on the PPG data.

Illustrative Embodiment 2. The method of training the AI model of illustrative embodiment 1, wherein the training includes training the AI model, using the training data, to determine one or more loss functions, and training the AI model, using the one or more loss functions, to determine the one or more outputs.

Illustrative Embodiment 3. The method of training the AI model of any one of illustrative embodiments 1-2, wherein the AI model is configured such that the one or more outputs include a first output, a second output, and a third output, the one or more loss functions include a first loss function, a second loss function, and a third loss function, the first loss function is based on a first true value and the first output, the second loss function is based on a second true value and the second output, and the third loss function is based on a third true value and the third output.

Illustrative Embodiment 4. The method of training the AI model of any one of illustrative embodiments 1-3, wherein the training the AI model, using the one or more loss functions, to determine the one or more outputs includes continuously adjusting parameters of the plurality of machine learning models until the first output equals the first true value, the second output equals the second true value, and the third output equals the third true value.

Illustrative Embodiment 5. The method of training the AI model of any one of illustrative embodiments 1-4, wherein the first output is based on data representing an inflection point area ratio (IPA) morphology metric of the PPG data, the second output is based on data representing a stress-induced vascular response index (sVRI) morphology metric of the PPG data, and the third output is based on data representing a signal quality index (SQI) morphology metric of the PPG data.

Illustrative Embodiment 6. The method of training the AI model of illustrative embodiment 1, wherein the plurality of machine learning models are configured to receive the PPG data and output the one or more outputs, the one or more outputs including a first output, a second output, and a third output.

Illustrative Embodiment 7. The method of training the AI model of any one of illustrative embodiment 1 and/or 6, wherein the plurality of machine learning models include, a first machine learning model including a first neural network configured to receive the PPG data and output the first output, a second machine learning model including a second neural network configured to receive the PPG data and output the second output, and a third machine learning model including a third neural network configured to receive the PPG data and output the third output.

Illustrative Embodiment 8. The method of training the AI model of any one of illustrative embodiments 1, 6, and/or 7, wherein the training the AI model, using the training data, to determine one or more outputs based on the PPG data includes, receiving, by an encoder, the PPG data including the PPG signals, processing, by the encoder, the PPG data to generate and output a first embedding of PPG data, generating, by the first machine learning model, the first output based on the first embedding of PPG data, processing, by the second machine learning model, the first embedding of PPG data to generate a second embedding of PPG data, generating, by the second machine learning model, the second output based on the second embedding of PPG data, and generating, by the third machine learning model, the third output based on the first embedding of PPG data.

Illustrative Embodiment 9. The method of training the AI model of any one of illustrative embodiments 1, 6, 7, and/or 8, wherein the method further includes processing the PPG data to filter noise in the PPG signals before generating the first embedding of PPG data.

Illustrative embodiment 10. The method of training the AI model of illustrative embodiment 1, wherein the AI model is configured to link the PPG data to one or more of health conditions based on processing the PPG data.

Illustrative embodiment 11. A processing device including a memory storing a program of instructions, and a processor coupled to the memory, the processor configured to execute the program of instructions to implement an artificial intelligence (AI) model trained on training data including Photoplethysmography (PPG) data based on PPG signals, the AI model including a plurality of machine learning models and trained to, receive the PPG data including the PPG signals; and generate an output corresponding to the PPG data.

Illustrative embodiment 12. The processing device of illustrative embodiment 11, wherein the AI model is trained to determine one or more morphology metrics of the PPG data, and generate the output based on the one or more morphology metrics of the PPG data.

Illustrative embodiment 13. The processing device of any one of illustrative embodiments 11-12, wherein the one or more morphology metrics of the PPG data includes an inflection point area ratio (IPA) morphology metric of the PPG data, a stress-induced vascular response index (sVRI) morphology metric of the PPG data, and a signal quality index (SQI) morphology metric of the PPG data.

Illustrative embodiment 14. The processing device of illustrative embodiment 11, wherein training the AI model includes receiving, by an encoder, the PPG data including the PPG signals, processing, by the encoder, the PPG data to generate and output a first embedding of PPG data, generating, by a first machine learning model, a first output and a first loss function, the first output based on the first embedding of PPG data, generating, by a second machine learning model, a second output and a second loss function, the second output based on a second embedding of PPG data, the second embedding of PPG data generated by the second machine learning model based on the first embedding of PPG data, and generating, by a third machine learning model, a third output and a third loss function, the third output based on the first embedding of PPG data, wherein the first loss function is based on a first true value and the first output, the second loss function is based on a second true value and the second output, and the third loss function is based on a third true value and the third output.

Illustrative embodiment 15. The processing device of any one of illustrative embodiments 11 and/or 14, wherein the training the AI model further includes continuously adjusting parameters of the plurality of machine learning models until the first output equals the first true value, the second output equals the second true value, and the third output equals the third true value.

Illustrative embodiment 16. The processing device of any one of illustrative embodiments 11, 14, and/or 15, wherein the first true value is a calculated IPA morphology metric value, the second true value is a calculated sVRI morphology metric value, the third true value is a calculated SQI morphology metric value, the first output is an estimated IPA morphology metric, the second output is an estimated sVRI morphology metric, and the third output is an estimated SQI morphology metric

Illustrative embodiment 17. The processing device of any one of illustrative embodiments 11, 14, 15, and/or 16, wherein the program of instructions includes instructions to cause the processor to re-train the AI model.

Illustrative embodiment 18. The processing device of illustrative embodiment 11, wherein each machine learning model of the plurality of machine learning models is configured to respectively determine one of an IPA morphology metric of the PPG data, a sVRI morphology metric of the PPG data, or a SQI morphology metric of the PPG data.

Illustrative embodiment 19. A method including providing, as an input, Photoplethysmography (PPG) data to an AI model, the AI model including a plurality of machine learning models and the PPG data including PPG signals obtained from an individual, processing, by the plurality of machine learning models, the PPG data to determine one or more morphology metrics, and outputting, by the AI model, a statistical value based on the one or more morphology metrics.

Illustrative embodiment 20. The method of illustrative embodiment 20 further including further including linking, by the AI model, the statistical value to one or more health conditions of the individual based on the one or more morphology metrics, wherein the one or more morphology metrics include, an inflection point area ratio (IPA) morphology metric of the PPG data, a stress-induced vascular response index (sVRI) morphology metric of the PPG data, and a signal quality index (SQI) morphology metric of the PPG data.

Benefits, other advantages, and solutions to problems have been described above with regard to specific embodiments of the invention. However, the benefits, advantages, solutions to problems, and any element(s) that may cause or result in such benefits, advantages, or solutions, or cause such benefits, advantages, or solutions to become more pronounced are not to be construed as a critical, required, or essential feature or element of any or all the claims.

The foregoing description of the embodiments has been provided for purposes of illustration and description. It is not intended to be exhaustive or to limit the disclosure. Individual elements or features of a particular embodiment are generally not limited to that particular example embodiment but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of training an artificial intelligence (AI) model, the method comprising:
providing training data to the AI model, the AI model including a plurality of machine learning models and the training data including Photoplethysmography (PPG) data based on PPG signals obtained from a plurality of individuals; and
training the AI model, using the training data, to determine one or more outputs based on the PPG data.

2. The method of claim 1, wherein the training comprises:
training the AI model, using the training data, to determine one or more loss functions; and
training the AI model, using the one or more loss functions, to determine the one or more outputs.

3. The method of claim 2, wherein the AI model is configured such that
the one or more outputs include a first output, a second output, and a third output,
the one or more loss functions include a first loss function, a second loss function, and a third loss function,
the first loss function is based on a first true value and the first output,
the second loss function is based on a second true value and the second output, and
the third loss function is based on a third true value and the third output.

4. The method of claim 3, wherein the training the AI model, using the one or more loss functions, to determine the one or more outputs includes continuously adjusting parameters of the plurality of machine learning models until the first output equals the first true value, the second output equals the second true value, and the third output equals the third true value.

5. The method of claim 4, wherein
the first output is based on data representing an inflection point area ratio (IPA) morphology metric of the PPG data,
the second output is based on data representing a stress-induced vascular response index (sVRI) morphology metric of the PPG data, and
the third output is based on data representing a signal quality index (SQI) morphology metric of the PPG data.

6. The method of claim 1, wherein the plurality of machine learning models are configured to receive the PPG data and output the one or more outputs, the one or more outputs including a first output, a second output, and a third output.

7. The method of claim 6, wherein the plurality of machine learning models include
a first machine learning model including a first neural network configured to receive the PPG data and output the first output,
a second machine learning model including a second neural network configured to receive the PPG data and output the second output, and
a third machine learning model including a third neural network configured to receive the PPG data and output the third output.

8. The method of claim 7, wherein the training the AI model, using the training data, to determine one or more outputs based on the PPG data includes
receiving, by an encoder, the PPG data including the PPG signals,
processing, by the encoder, the PPG data to generate and output a first embedding of PPG data,
generating, by the first machine learning model, the first output based on the first embedding of PPG data,
processing, by the second machine learning model, the first embedding of PPG data to generate a second embedding of PPG data,
generating, by the second machine learning model, the second output based on the second embedding of PPG data, and
generating, by the third machine learning model, the third output based on the first embedding of PPG data.

9. The method of claim 8, further comprising:
processing the PPG data to filter noise in the PPG signals before generating the first embedding of PPG data.

10. The method of claim 1, wherein the AI model is configured to link the PPG data to one or more of health conditions based on processing the PPG data.

11. A processing device comprising:
a memory storing a program of instructions; and
a processor coupled to the memory, the processor configured to execute the program of instructions to implement an artificial intelligence (AI) model trained on training data including Photoplethysmography (PPG) data based on PPG signals, the AI model including a plurality of machine learning models and trained to
receive the PPG data including the PPG signals; and
generate an output corresponding to the PPG data.

12. The processing device of claim 11, wherein the AI model is trained to
determine one or more morphology metrics of the PPG data, and
generate the output based on the one or more morphology metrics of the PPG data.

13. The processing device of claim 12, wherein the one or more morphology metrics of the PPG data includes
an inflection point area ratio (IPA) morphology metric of the PPG data,
a stress-induced vascular response index (sVRI) morphology metric of the PPG data, and
a signal quality index (SQI) morphology metric of the PPG data.

14. The processing device of claim 11, wherein the training the AI model includes
receiving, by an encoder, the PPG data including the PPG signals,
processing, by the encoder, the PPG data to generate and output a first embedding of PPG data,
generating, by a first machine learning model, a first output and a first loss function, the first output based on the first embedding of PPG data,
generating, by a second machine learning model, a second output and a second loss function, the second output based on a second embedding of PPG data, the second embedding of PPG data generated by the second machine learning model based on the first embedding of PPG data, and
generating, by a third machine learning model, a third output and a third loss function, the third output based on the first embedding of PPG data, wherein
the first loss function is based on a first true value and the first output,
the second loss function is based on a second true value and the second output, and
the third loss function is based on a third true value and the third output.

15. The processing device of claim 14, wherein the training the AI model further includes continuously adjusting parameters of the plurality of machine learning models until the first output equals the first true value, the second output equals the second true value, and the third output equals the third true value.

16. The processing device of claim 15, wherein
the first true value is a calculated IPA morphology metric value,
the second true value is a calculated sVRI morphology metric value,
the third true value is a calculated SQI morphology metric value,
the first output is an estimated IPA morphology metric,
the second output is an estimated sVRI morphology metric, and
the third output is an estimated SQI morphology metric.

17. The processing device of claim 16, wherein the program of instructions includes instructions to cause the processor to re-train the AI model.

18. The processing device of claim 11, wherein each machine learning model of the plurality of machine learning models is configured to respectively determine one of an IPA morphology metric of the PPG data, a sVRI morphology metric of the PPG data, or a SQI morphology metric of the PPG data.

19. A method comprising:
providing, as an input, Photoplethysmography (PPG) data to an AI model, the AI model including a plurality of machine learning models and the PPG data including PPG signals obtained from an individual;
processing, by the plurality of machine learning models, the PPG data to determine one or more morphology metrics; and
outputting, by the AI model, a statistical value based on the one or more morphology metrics.

20. The method of claim 19, further comprising:
linking, by the AI model, the statistical value to one or more health conditions of the individual based on the one or more morphology metrics,
wherein the one or more morphology metrics include
an inflection point area ratio (IPA) morphology metric of the PPG data,
a stress-induced vascular response index (sVRI) morphology metric of the PPG data, and
a signal quality index (SQI) morphology metric of the PPG data.
